(19) United States
(12) Patent Application Publication
(10) Pub. No.: US 2026/0108386 A1
(43) Pub. Date: Apr. 23, 2026

(54) OCULAR IMPLANT, OCULAR IMPLANT DELIVERY SYSTEM, AND METHODS OF DEPLOYING AN OCULAR IMPLANT

(71) Applicant: Alcon Inc., Fribourg (CH)

(72) Inventors: David S. KIMBALL, Irvine, CA (US); Jestwin E. LEE, IV, Grandview, TX (US)

(73) Assignee: Alcon Inc., Fribourg (CH)

(21) Appl. No.: 19/320,393

(22) Filed: Sep. 5, 2025

Related U.S. Application Data
(60) Provisional application No. 63/708,641, filed on Oct. 17, 2024.

(51) Int. Cl. A61F 9/00; A61F 9/007

(52) U.S. Cl. CPC A61F 9/0017; A61F 9/00781

(57) ABSTRACT

Disclosed are ocular implants, ocular implant delivery systems, and methods of deploying an ocular implant. For example, the ocular implant can comprise a plurality of supporting frames and spines connecting the supporting frames. The ocular implant can comprise a distal supporting frame configured as an open trough having a trough nadir at a distal end. The trough nadir can be positioned radially outward of the spines. The ocular implant can also comprise a proximal portion comprising a pair of tool engagement ears curled laterally inward toward one another. A delivery system used to deploy the ocular implant can comprise a cannula comprising a distal portion having a proximal curved bend and a distal curved bend. The proximal curved bend can be bent in a different direction than the distal curved bend. The delivery system can also comprise a camera coupled to the cannula.

### CROSS-REFERENCE TO RELATED APPLICATION

This application claims the benefit of priority to U.S. Provisional Patent Application No. 63/708,641 filed on Oct. 17, 2024, the content of which is incorporated herein by reference in its entirety.

### TECHNICAL FIELD

The present disclosure relates generally to the field of glaucoma management devices and, more specifically, to an ocular implant, ocular implant delivery systems, and methods of deploying an ocular implant.

### BACKGROUND

According to a draft report by The National Eye Institute (NEI) at The United States National Institutes of Health (NIH), glaucoma is now the leading cause of irreversible blindness worldwide and the second leading cause of blindness, behind cataract, in the world. Thus, the NEI draft report concludes, “it is critical that significant emphasis and resources continue to be devoted to determining the pathophysiology and management of this disease.” Glaucoma researchers have found a strong correlation between high intraocular pressure and glaucoma. For this reason, eye care professionals routinely screen patients for glaucoma by measuring intraocular pressure using a device known as a tonometer. Many modern tonometers make this measurement by blowing a sudden puff of air against the outer surface of the eye.

The eye can be conceptualized as a ball filled with fluid. There are two types of fluid inside the eye. The cavity behind the lens is filled with a viscous fluid known as vitreous humor. The cavities in front of the lens are filled with a fluid known as aqueous humor. Whenever a person views an object, he or she is viewing that object through both the vitreous humor and the aqueous humor.

Whenever a person views an object, he or she is also viewing that object through the cornea and the lens of the eye. In order to be transparent, the cornea and the lens can include no blood vessels. Accordingly, no blood flows through the cornea and the lens to provide nutrition to these tissues and to remove waste from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the anterior chamber of the eye through the trabecular meshwork and into Schlemm's canal as new aqueous humor is secreted by the epithelial cells of the ciliary body. This excess aqueous humor enters the venous blood stream from Schlemm's canal and is carried along with the venous blood leaving the eye.

When the natural drainage mechanisms of the eye stop functioning properly, the pressure inside the eye begins to rise. Researchers have theorized that prolonged exposure to high intraocular pressure causes damage to the optic nerve that transmits sensory information from the eye to the brain. This damage to the optic nerve results in loss of peripheral vision. As glaucoma progresses, more and more of the visual field is lost until the patient is completely blind.

In addition to drug treatments, a variety of surgical treatments for glaucoma have been performed including ocular implants implanted to increase aqueous humor outflow. However, there can be challenges associated with the proper placement of such ocular implants within the eye of a patient.

### SUMMARY

Disclosed herein are ocular implants, ocular implant delivery systems, and methods of deploying an ocular implant. One aspect of the disclosure concerns an ocular implant comprising an implant body extending in a curved configuration and configured to lower an intraocular pressure of an eye when implanted within Schlemm's canal of the eye. The implant body can form an arc. The implant body can comprise a distal portion having a distal end and a proximal portion having a proximal end, a plurality of openings disposed in between the distal end and the proximal end, and a plurality of supporting frames and spines connecting the supporting frames. Each of the openings can be surrounded by at least one of the supporting frames. The distal portion can comprise a distal supporting frame configured as an open trough having a trough nadir at the distal end. The trough nadir can be positioned radially outward of the spines.

In some embodiments, the distal supporting frame can comprise a first distal frame strut and a second distal frame strut. The first distal frame strut and the second distal frame strut can form part of the open trough. For example, the first distal frame strut and the second distal frame strut can form sides of the open trough.

In some embodiments, the open trough can have a trough concavity. The trough concavity can face a trabecular meshwork of the eye when the ocular implant is implanted within Schlemm's canal.

In some embodiments, the distal portion can comprise a distal opening surrounded by the distal supporting frame. The distal opening can comprise an opening distal end and an opening proximal end. The opening distal end of the distal opening can be positioned radially outward of the opening proximal end of the distal opening.

In some embodiments, the openings comprise a distal opening surrounded by the distal supporting frame and an intermediate opening positioned proximal to the distal opening. The distal opening can be defined by a distal opening contour. The intermediate opening can be defined by an intermediate opening contour. The distal opening contour can be shaped differently from the intermediate opening contour.

In some embodiments, the supporting frames can further comprise an intermediate supporting frame positioned proximally of the distal supporting frame. A shape of the intermediate supporting frame can be different from the shape of the distal supporting frame.

In some embodiments, the proximal portion can comprise a substantially-tubular portion and a pair of tool engagement ears coupled to the substantially-tubular portion by a narrow connecting portion. The pair of tool engagement ears can be curled laterally inward toward one another. The proximal portion can be configured to interlock with a complementary portion of a delivery tool.

In some embodiments, the substantially-tubular portion can have an elongated gap extending along a length of the substantially-tubular portion. The substantially-tubular portion can define a tubular-shaped lumen extending therethrough.

In some embodiments, the pair of tool engagement ears comprise a first ear terminal end and a second ear terminal end. The first ear terminal end can be separated from the second ear terminal end by a minimum separation distance. The tubular-shaped lumen can have a lumen diameter. The minimum separation distance can be less than the lumen diameter.

In some embodiments, the narrow connecting portion can have a minimum connecting portion width. The minimum connecting portion width can be smaller than the minimum separation distance and the lumen diameter.

In some embodiments, each of the tool engagement ears can comprise an ear radius of curvature. The substantially-tubular portion can comprise a tube radius of curvature. The ear radius of curvature can be different from the tube radius of curvature.

In some embodiments, the pair of tool engagement ears are curled laterally inward toward a medial line bisecting the proximal portion.

In some embodiments, the ocular implant can be configured to be deployed out of a cannula. The cannula can comprise a proximal curved bend and a distal curved bend. The proximal curved bend can be bent in a different direction than the distal curved bend.

In some embodiments, the distal curved bend can have a bend radius of curvature of between 3.0 mm and 4.0 mm. The proximal curved bend can have a bend radius of curvature of between 3.0 mm and 4.0 mm.

In some embodiments, the ocular implant can be configured to be deployed out of a cannula. The cannula can comprise a camera coupled to at least part of the cannula.

Also disclosed is an ocular implant comprising an implant body extending in a curved configuration and configured to lower an intraocular pressure of an eye when implanted within Schlemm's canal of the eye. The implant body can form an arc. The implant body can comprise a proximal portion having a proximal end and a distal portion having a distal end. The implant body can also comprise a plurality of openings disposed in between the distal end and the proximal end and a plurality of supporting frames and spines connecting the supporting frames. Each of the openings can be surrounded by at least one of the supporting frames. The proximal portion can comprise a substantially-tubular portion and a pair of tool engagement ears coupled to the substantially-tubular portion by a narrow connecting portion. The pair of tool engagement ears can be curled laterally inward toward one another. The proximal portion can be configured to interlock with a complementary portion of a delivery tool.

In some embodiments, the substantially-tubular portion can have an elongated gap extending along a length of the substantially-tubular portion. The substantially-tubular portion can define a tubular-shaped lumen extending therethrough.

In some embodiments, the pair of tool engagement ears can comprise a first ear terminal end and a second ear terminal end. The first ear terminal end can be separated from the second ear terminal end by a minimum separation distance. The tubular-shaped lumen can have a lumen diameter. The minimum separation distance can be less than the lumen diameter.

In some embodiments, the narrow connecting portion can have a minimum connecting portion width. The minimum connecting portion width can be smaller than the minimum separation distance and the lumen diameter.

In some embodiments, each of the tool engagement ears can comprise an ear radius of curvature. The substantially-tubular portion can comprise a tube radius of curvature. The ear radius of curvature can be different from the tube radius of curvature.

In some embodiments, the pair of tool engagement ears are curled laterally inward toward a medial line bisecting the proximal portion.

In some embodiments, the distal portion can comprise a distal supporting frame configured as an open trough having a trough nadir at the distal end.

In some embodiments, the trough nadir is positioned radially outward of the spines.

In some embodiments, the distal supporting frame can comprise a first distal frame strut and a second distal frame strut. The first distal frame strut and the second distal frame strut can form part of the open trough. For example, the first distal frame strut and the second distal frame strut can form sides of the open trough.

In some embodiments, the open trough can have a trough concavity. The trough concavity can face a trabecular meshwork of the eye when the ocular implant is implanted within Schlemm's canal.

In some embodiments, the distal portion can comprise a distal opening surrounded by the distal supporting frame. The distal opening can comprise an opening distal end and an opening proximal end. The opening distal end of the distal opening can be radially outward of the opening proximal end of the distal opening.

In some embodiments, the openings can comprise a distal opening surrounded by the distal supporting frame and an intermediate opening positioned proximal to the distal opening. The distal opening can be defined by a distal opening contour. The intermediate opening can be defined by an intermediate opening contour. The distal opening contour can be shaped differently from the intermediate opening contour.

In some embodiments, the supporting frames can further comprise an intermediate supporting frame positioned proximally of the distal supporting frame. A shape of the intermediate supporting frame is different from the shape of the distal supporting frame.

Also disclosed is a delivery system for deploying an ocular implant. The delivery system can comprise a system housing and a cannula extending from the system housing. The cannula can comprise a cannula distal portion comprising a proximal curved bend and a distal curved bend. The proximal curved bend can be bent in a different direction than the distal curved bend. The delivery system can also comprise a delivery tool housed at least partially within the cannula and translatable therethrough. The delivery tool can be configured to detachably engage with an ocular implant. The ocular implant can comprise an implant body extending in a curved configuration and configured to lower an intraocular pressure of an eye when implanted within Schlemm's canal of the eye. The implant body can comprise a distal portion having a distal end and a proximal portion having a proximal end. The implant body can also comprise a plurality of openings disposed in between the distal end and the proximal end, a plurality of supporting frames, and spines connecting the supporting frames. Each of the openings can be surrounded by at least one of the supporting frames.

In some embodiments, the distal curved bend can have a bend radius of curvature of between 3.0 mm and 4.0 mm. The proximal curved bend can have a bend radius of curvature of between 3.0 mm and 4.0 mm.

In some embodiments, the cannula can comprise a camera coupled to at least part of the cannula.

In some embodiments, the cannula can be shaped substantially as a shepherd's hook.

In some embodiments, the ocular implant can comprise a proximal portion comprising a substantially-tubular portion and a pair of tool engagement ears coupled to the substantially-tubular portion by a narrow connecting portion. The pair of tool engagement ears can be curled laterally inward toward one. The delivery tool can comprise a tool distal portion configured to interlock with the proximal portion of the ocular implant.

In some embodiments, the pair of tool engagement ears can be curled laterally inward toward a medial line bisecting the proximal portion.

In some embodiments, each of the tool engagement ears can comprise an ear radius of curvature. The substantially-tubular portion can comprise a tube radius of curvature. The ear radius of curvature can be different from the tube radius of curvature.

Also disclosed is another embodiment of the delivery system for deploying an ocular implant. The delivery system can comprise a system housing, a cannula extending from the system housing; a camera coupled to the cannula, and a delivery tool housed at least partially within the cannula and translatable therethrough. The delivery tool can be configured to detachably engage with an ocular implant. The ocular implant can comprise an implant body extending in a curved configuration and configured to lower an intraocular pressure of an eye when implanted within Schlemm's canal of the eye. The implant body can comprise a distal portion having a distal end and a proximal portion having a proximal end, a plurality of openings disposed in between the distal end and the proximal end, and a plurality of supporting frames and spines connecting the supporting frames. Each of the openings can be surrounded by at least one of the supporting frames.

In some embodiments, the camera can be coupled to a substantially straight shaft of the cannula.

In some embodiments, a lens of the camera can be positioned to capture a deployment of the ocular implant out of a distal opening of the cannula.

In some embodiments, the camera can be coupled to an exterior surface of the cannula.

In some embodiments, the cannula can comprise a cannula distal portion comprising a proximal curved bend and a distal curved bend. The proximal curved bend can be bent in a different direction than the distal curved bend.

In some embodiments, the distal curved bend can have a bend radius of curvature of between 3.0 mm and 4.0 mm. The proximal curved bend can have a bend radius of curvature of between 3.0 mm and 4.0 mm.

In some embodiments, the distal portion of the cannula can be shaped substantially as a shepherd's hook.

In some embodiments, the camera is communicatively coupled to a computing device via a wired cable connection.

In some embodiments, the camera can have a field-of-view of between about 100 degrees and 130 degrees.

In some embodiments, the camera can have a camera viewing range of between about 3.0 mm and 30 mm.

In some embodiments, the camera can comprise a complementary metal-oxide-semiconductor (CMOS) image sensor.

In some embodiments, the CMOS image sensor can have a sensor width of about 0.70 mm.

In some embodiments, the camera can comprise a glass camera lens.

Also disclosed is a method of deploying an ocular implant. The method can comprise inserting a part of a cannula through an incision in an eye, placing a distal opening of the cannula into fluid communication with Schlemm's canal of the eye, and advancing an implant body of the ocular implant through a cannula distal portion using a delivery tool detachably coupled to the ocular implant. The cannula distal portion can comprise a proximal curved bend and a distal curved bend. The proximal curved bend can be bent in a different direction than the distal curved bend. The method can also comprise advancing the ocular implant out of the distal opening of the cannula and into Schlemm's canal.

Further disclosed is another method of deploying an ocular implant. The method can comprise inserting a part of a cannula through an incision in an eye, placing a distal opening of the cannula into fluid communication with Schlemm's canal of the eye, advancing an implant body of the ocular implant out of a distal opening of the cannula and into Schlemm's canal, and capturing a video of the ocular implant advancing out of the distal opening of the cannula using a camera coupled to the cannula.

### BRIEF DESCRIPTION OF THE DRAWINGS

FIG. 1 is a stylized perspective view of a portion of a human eye and part of an ocular implant disposed in Schlemm's canal.

FIG. 2A is a perspective view of one embodiment of the ocular implant.

FIG. 2B is a perspective view of a volume defined by an implant body of the ocular implant of FIG. 2A.

FIG. 3A is a perspective view of one embodiment of a delivery system used to deliver the ocular implant.

FIG. 3B is an enlarged view of a distal portion of a cannular of the delivery system and the ocular implant.

FIG. 3C illustrates a delivery tool of the delivery system extending partially out of a distal opening of the cannula.

FIG. 4 is a perspective view of a distal segment of one embodiment of a cannula of the delivery system comprising a camera coupled to the cannula.

FIG. 5A is a close-up perspective view of a proximal portion of one embodiment of the ocular implant.

FIG. 5B is a close-up front view of the proximal portion of the ocular implant.

FIG. 6 is a stylized representation of part of a procedure for deploying the ocular implant.

FIG. 7 is another stylized representation of part of the procedure for deploying the ocular implant.

### DETAILED DESCRIPTION

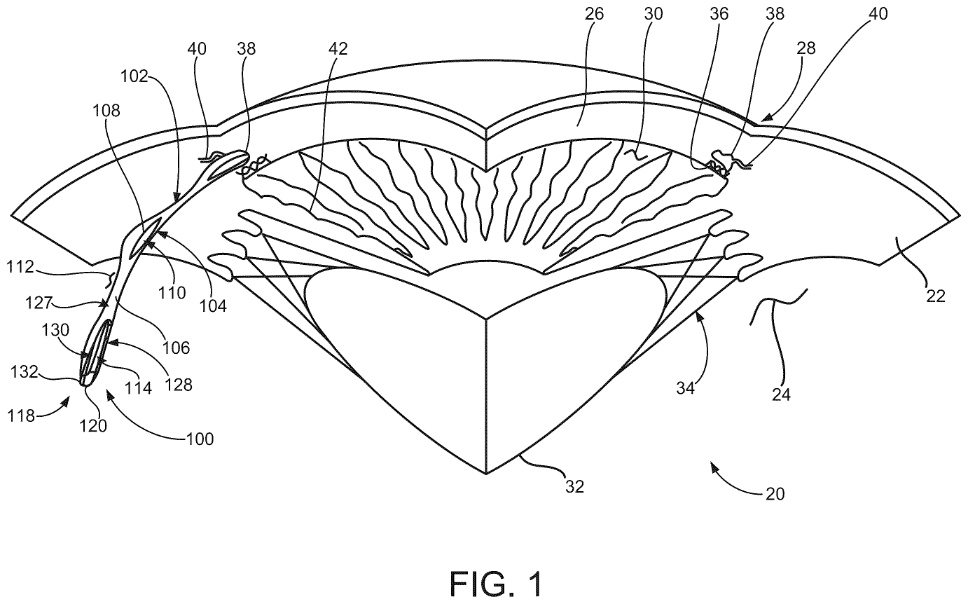

FIG. 1 is a stylized perspective view depicting a portion of a human eye 20 and a portion of an ocular implant 100 disposed in Schlemm's canal. The human eye 20 can be conceptualized as a fluid-filled ball having two chambers. Sclera 22 of eye 20 surrounds a posterior chamber 24 filled with a viscous fluid known as vitreous humor. Cornea 26 of eye 20 encloses an anterior chamber 30 that is filled with a fluid known as aqueous humor. The cornea 26 meets the sclera 22 at a limbus 28 of eye 20. A lens 32 of eye 20 is located between anterior chamber 30 and posterior chamber 24. Lens 32 is held in place by a number of ciliary zonules 34. Whenever a person views an object, he or she is viewing that object through the cornea, the aqueous humor, and the lens of the eye. In order to be transparent, the cornea and the lens can include no blood vessels. Accordingly, no blood flows through the cornea and the lens to provide nutrition to these tissues and to remove waste from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the eye as new aqueous humor is secreted by the epithelial cells of the ciliary body. This excess aqueous humor enters the blood stream and is carried away by venous blood leaving the eye. In a healthy eye, aqueous humor flows out of the anterior chamber 30 through the trabecular meshwork 36 and into Schlemm's canal 38, located at the outer edge of the iris 42. Aqueous humor exits Schlemm's canal 38 by flowing through a number of outlets 40. After leaving Schlemm's canal 38, aqueous humor is absorbed into the venous blood stream.

As shown in FIG. 1, the ocular implant 100 can have a substantially tubular implant body 102 comprising a plurality of tissue supporting frames 104 and a plurality of spines 106. Each of the tissue supporting frames 104 can comprise edges 108 that define openings 110. The openings 110 can be formed as elongate slots or apertures in fluid communication with an elongate channel 112 defined by an inner surface 114 of the implant body 102. The openings 110 and the elongate channel 112 can allow aqueous humor to flow laterally across and/or through the ocular implant 100 when the ocular implant 100 is implanted within Schlemm's canal.

The ocular implant 100 can be inserted into Schlemm's canal of a human eye to facilitate the flow of aqueous humor out of the anterior chamber. This flow can include axial flow along Schlemm's canal, flow from the anterior chamber into Schlemm's canal, and flow leaving Schlemm's canal via outlets communicating with Schlemm's canal. When in place within the eye, ocular implant 100 can support trabecular mesh tissue and Schlemm's canal tissue and will provide for improved communication between the anterior chamber and Schlemm's canal (via the trabecular meshwork) and between pockets or compartments along Schlemm's canal. As shown in FIG. 1, the ocular implant 100 is implanted in a manner such that the channel 112 opens in a radially outward direction within Schlemm's canal.

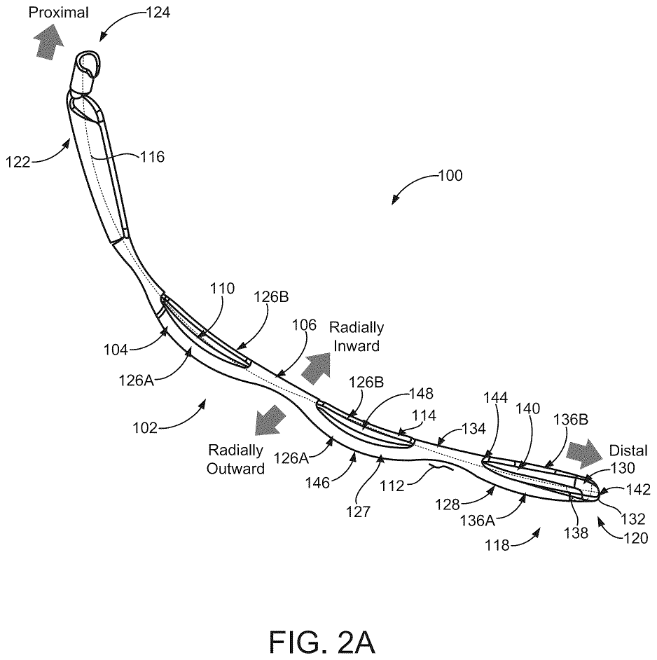

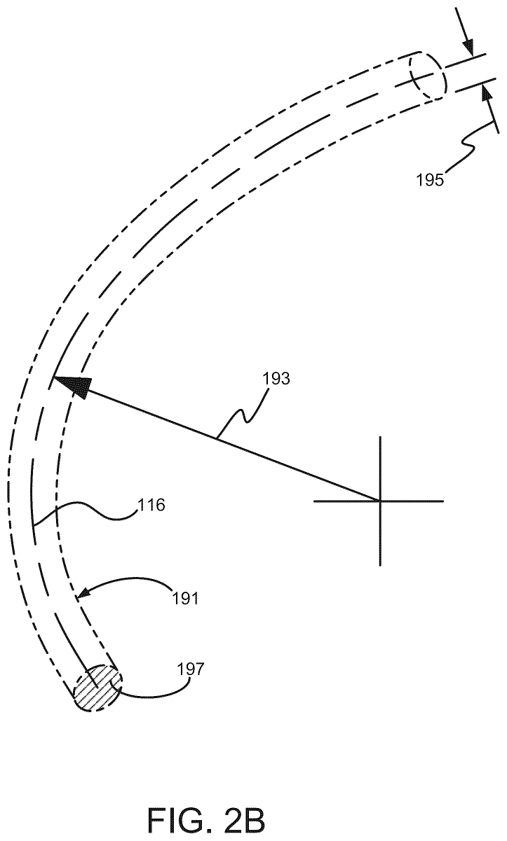

FIG. 2A is a perspective view of one embodiment of the ocular implant 100. The ocular implant 100 can comprise an implant body 102 that extends in a curved arc-shaped configuration such that a curved longitudinal axis 116 of the implant body 102 also forms an arc. The ocular implant 100 can lower an intraocular pressure of an eye of a subject when implanted within Schlemm's canal of the subject's eye.

The implant body 102 can comprise a distal portion 118 having a distal end 120 and a proximal portion 122 having a proximal end 124. The implant body 102 can also comprise a plurality of openings 110 disposed in between the distal end 120 and the proximal end 124.

The implant body 102 can further comprise a plurality of tissue supporting frames 104 and a plurality of spines 106. As shown in FIG. 2A, the spines 106 and tissue supporting frames 104 can be arranged in a repeating AB pattern in which each A is a tissue supporting frame 104 and each B is a spine 106. In the embodiment of FIG. 2A, one spine 106 can extend between each adjacent pair of tissue supporting frames 104.

Each of the openings 110 can be surrounded by at least one of the tissue supporting frames 104. The tissue supporting frames 104 can be made up of frame struts. For example, at least one of the tissue supporting frames 104 can be made up of a first frame strut 126A and a second frame strut 126B.

In some embodiments, the frame struts can be curved in a radially outward direction such that the sides of the implant body 102 have an undulating or wave-like shape or profile along a length of the implant body 102.

The distal portion 118 of the implant body 102 can comprise a distal supporting frame 128. The distal supporting frame 128 can be configured as an open trough 130 having a trough nadir 132 at the distal end 120 of the implant body 102.

For example, as depicted in FIG. 2A, at least part of the open trough 130 can be in the shape of a parabolic trough. The open trough 130 can converge in a direction of the distal end 120 and form a rounded trough open end. The rounded trough open end can be shaped substantially as a rounded scoop or rounded trowel.

As shown in FIG. 2A, the trough nadir 132 of the open trough 130 can be positioned or formed radially outward of the spines 106. For example, the trough nadir 132 or the lowest point along the open trough 130 can be positioned or formed radially outward of a distal spine 134 of the implant body 102.

The distal supporting frame 128 can comprise a first distal frame strut 136A and a second distal frame strut 136B. The first distal frame strut 136A and the second distal frame strut 136B can make up part of the open trough 130 (e.g., the sides of the open trough 130). The distal supporting frame 128 can be cut into the shape of an open trough from a flexible and biocompatible tube (e.g., a Nitinol tube) serving as the starting material.

As shown in FIG. 2A, the open trough 130 can have a trough concavity 138. The trough concavity 138 can face the trabecular meshwork 36 of the eye 20 (see FIG. 1) when the ocular implant 100 is implanted within Schlemm's canal.

The distal portion 118 can further comprise a distal opening 140 surrounded by the distal supporting frame 128. The distal opening 140 can be in fluid communication with the elongate channel 112 and the other openings 110 of the ocular implant 100.

The distal opening 140 can comprise an opening distal end 142 and an opening proximal end 144. The opening proximal end 144 can serve as a distal end of the distal spine 134. The opening distal end 142 of the distal opening 140 can be positioned radially outward of the opening proximal end 144 of the distal opening 140.

The opening proximal end 144 of the distal opening 140 can also be considered to be positioned above or positioned dorsal to the opening distal end 142 (which can be considered to be positioned below or ventral to the opening proximal end 144).

As shown in FIG. 2A, the tissue supporting frames 104 can comprise an intermediate supporting frame 146 positioned proximally of the distal supporting frame 128. The overall shape or contour of the intermediate supporting frame 146 can be different from the overall shape or contour of the distal supporting frame 128.

For example, the intermediate supporting frame 146 (as well as the other tissue supporting frames 104 other than the distal supporting frame 128) can be substantially shaped as an elongated ellipse or oval with the curved elongated lateral sides of the ellipse or oval bent in a radially outward direction. The curved elongated lateral sides of the ellipse or oval can be bent along the longitudinal axis 116 bisecting the ellipse or oval.

In some embodiments, the intermediate supporting frame 146 (as well as the other tissue supporting frames 104 other than the distal supporting frame 128) can be substantially shaped as a curved saddle having an elongated hole or opening in the middle of the curved saddle.

The intermediate supporting frame 146 can also surround and define an intermediate opening 148 located proximally of the distal opening 140. The distal opening 140 can be defined by a distal opening contour and the intermediate opening 148 can be defined by an intermediate opening contour. The intermediate opening contour can be shaped differently from the distal opening contour.

For example, the intermediate opening contour can be shaped substantially as an elongated oval or elongated ellipse while the distal opening contour can be shaped substantially as an elongated U.

In some embodiments, the openings 110 of the ocular implant 100 can be referred to as windows. Since the opening distal end 142 of the distal opening 140 drops down or is positioned radially outward of the opening proximal end 144, the distal opening 140 can be considered a “reverse window” relative to the other windows or openings 110 of the ocular implant 100.

One technical problem faced by the applicant is how to design an ocular implant for implantation into Schlemm's canal where a distal end of the ocular implant does not inadvertently roll downward or in a radially inward direction toward the trabecular meshwork 36 (see, e.g., FIG. 1) and pierce the trabecular meshwork 36 or dive down into the ciliary body. One technical solution discovered and developed by the applicant is to design the ocular implant 100 such that the distal portion 118 of the ocular implant 100 comprises a distal supporting frame 128 configured as an open trough 130 having a trough nadir 132 at the distal end 120 of the ocular implant 100 that is positioned radially outward or behind the spines 106 of the ocular implant 100. When the distal portion 118 of the ocular implant 100 is implemented in this manner, the trough nadir 132 rides against the back wall of Schlemm's canal rather than the trabecular meshwork. The back wall of Schlemm's canal is the sclera, which is tougher than the trabecular meshwork.

The ocular implant 100 can be made from a metal, metal alloy, a polymer (some examples of which are disclosed below), a metal-polymer composite, a ceramic, combinations thereof, and the like, or other suitable material.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example, a superelastic nitinol can be used to achieve desired properties.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, utylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

The ocular implant 100 can further comprise a coating disposed on the inner surfaces 114 and/or outer surfaces 127 of the ocular implant 100. In some embodiments, the coating can cover only a portion of the inner surface 114 and/or the outer surfaces 127. For example, the coating can cover 10% or more, 25% or more, 50% or more, or 75% or more of the surface area of the ocular implant 100. In some instances, the coating can cover less than 10% or more than 75% of the surface area of the implant 100, as desired.

The coating can be formed of, or otherwise comprise, a therapeutic agent. In some embodiments, the coating can release the therapeutic agent. The coating can release the therapeutic agent controllably over a period of time. In some embodiments, the therapeutic agent can be applied directly to the ocular implant 100 while in other embodiments, the ocular implant can be dispersed within a matrix material. For example, the therapeutic agent can be dispersed within a biocompatible or biodegradable polymeric material. The concentration of therapeutic agent within the matrix material can vary depending on the desired treatment.

The biocompatible polymeric material used to form the bioactive agent-polymer composite layer(s) can include any polymeric material capable of forming a solidified composite layer in the presence of the bioactive material. The polymeric material of the present invention can be hydrophilic or hydrophobic, and is, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, polyolefins, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer can be a protein polymer, fibrin, collagen, and derivatives thereof, polysaccharides such as celluloses, starches, dextrans, alginates and derivatives of these polysaccharides, an extracellular matrix component, hyaluronic acid, or another biologic agent or a suitable mixture of any of these, for example.

The coating can include a single polymer or copolymer. The coating can also include copolymers or physical blends of any of the materials indicated above.

The therapeutic agents utilized with the ocular implant 100 can include one or more drugs provided below, either alone or in combination. The drugs utilized can also be the equivalent of, derivatives of, or analogs of one or more of the drugs provided below. The drugs can include but are not limited to pharmaceutical agents including anti-glaucoma medications, ocular agents, antimicrobial agents (e.g., antibiotic, antiviral, antiparasitic, antifungal agents), anti-inflammatory agents (including steroids or non-steroidal anti-inflammatory), biological agents including hormones, enzymes or enzyme-related components, antibodies or antibody-related components, oligonucleotides (including DNA, RNA, short-interfering RNA, antisense oligonucleotides, and the like), DNA/RNA vectors, viruses (either wild type or genetically modified) or viral vectors, peptides, proteins, enzymes, extracellular matrix components, and live cells configured to produce one or more biological components. The use of any particular drug is not limited to its primary effect or regulatory body-approved treatment indication or manner of use. Drugs also include compounds or other materials that reduce or treat one or more side effects of another drug or therapeutic agent. As many drugs have more than a single mode of action, the listing of any particular drug within any one therapeutic class below is only representative of one possible use of the drug and is not intended to limit the scope of its use with the ophthalmic implant system.

The therapeutic agents can be combined with any number of excipients as is known in the art. In addition to the biodegradable polymeric excipients discussed above, other excipients can be used, including, but not limited to, benzyl alcohol, ethylcellulose, methylcellulose, hydroxymethylcellulose, cetyl alcohol, croscarmellose sodium, dextrans, dextrose, fructose, gelatin, glycerin, monoglycerides, diglycerides, kaolin, calcium chloride, lactose, lactose monohydrate, maltodextrins, polysorbates, pregelatinized starch, calcium stearate, magnesium stearate, silicon dioxide, cornstarch, talc, and the like. The one or more excipients can be included in total amounts as low as about 1%, 5%, or 10% and, in other embodiments, can be included in total amounts as high as 50%, 70% or 90%.

Examples of drugs can include various anti-secretory agents; antimitotics and other anti-proliferative agents, including among others, anti-angiogenesis agents such as angiostatin, anecortave acetate, thrombospondin, VEGF receptor tyrosine kinase inhibitors and anti-vascular endothelial growth factor (anti-VEGF) drugs such as ranibizumab (LUCENTIS®) and bevacizumab (AVASTIN®), pegaptanib (MACUGEN®)sunitinib and sorafenib and any of a variety of known small-molecule and transcription inhibitors having anti-angiogenesis effect; classes of known ophthalmic drugs, including: glaucoma agents, such as adrenergic antagonists, including for example, beta-blocker agents such as atenolol propranolol, metipranolol, betaxolol, betaxolol hydrochloride carteolol, levobetaxolol, levobunolol, levobunolol hydrochloride, timolol, timolol hemihydrate, and timolol maleate; adrenergic agonists or sympathomimetic agents such as epinephrine, dipivefrin, clonidine, aparclonidine, and brimonidine; parasympathomimetics or cholingeric agonists such as pilocarpine, carbachol, phospholine iodine, and physostigmine, salicylate, acetylcholine chloride, eserine, diisopropyl fluorophosphate, demecarium bromide); muscarinics; carbonic anhydrase inhibitor agents, including topical and/or systemic agents, for example acetozolamide, brinzolamide, dorzolamide and methazolamide, ethoxzolamide, diamox, and dichlorphenamide; mydriatic-cycloplegic agents such as atropine, cyclopentolate, succinylcholine, homatropine, phenylephrine, scopolamine, and tropicamide; prostaglandins such as prostaglandin F2 alpha, antiprostaglandins, prostaglandin precursors, or prostaglandin analog agents such as bimatoprost, latanoprost, travoprost, tafluprost, and unoprostone; docosanoid compounds such as unoprostone.

Other examples of drugs can also include anti-inflammatory agents including for example glucocorticoids and corticosteroids such as betamethasone, cortisone, dexamethasone, dexamethasone 21-phosphate, methylprednisolone, prednisolone 21-phosphate, prednisolone acetate, prednisolone, fluroometholone, loteprednol, medrysone, fluocinolone acetonide, triamcinolone acetonide, triamcinolone, triamcinolone acetonide, beclomethasone, budesonide, flunisolide, fluorometholone, fluticasone, hydrocortisone, hydrocortisone acetate, loteprednol, rimexolone and non-steroidal anti-inflammatory agents including, for example, diclofenac, flurbiprofen, ibuprofen, bromfenac, nepafenac, and ketorolac, salicylate, indomethacin, ibuprofen, naxopren, piroxicam and nabumetone; anti-infective or antimicrobial agents such as antibiotics including, for example, tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, aminoglycosides such as gentamicin and tobramycin; fluoroquinolones such as ciprofloxacin, gatifloxacin, levofloxacin, moxifloxacin, norfloxacin, ofloxacin; bacitracin, erythromycin, fusidic acid, neomycin, polymyxin B, gramicidin, trimethoprim and sulfacetamide; antifungals such as amphotericin B and miconazole; antivirals such as idoxuridine trifluorothymidine, acyclovir, gancyclovir, interferon; antimicotics; immune-modulating agents such as antiallergenics, including, for example, sodium chromoglycate, antazoline, methapyriline, chlorpheniramine, cetrizine, pyrilamine, prophenpyridamine anti-histamine agents such as azelastine, emedastine and levocabastine; immunological drugs (such as vaccines and immune stimulants); MAST cell stabilizer agents such as cromolyn sodium, ketotifen, lodoxamide, nedocrimil, olopatadine and pemirolastciliary body ablative agents, such as gentimicin and cidofovir; and other ophthalmic agents such as verteporfin, proparacaine, tetracaine, cyclosporine and pilocarpine; inhibitors of cell-surface glycoprotein receptors; decongestants such as phenylephrine, naphazoline, tetrahydrazoline; lipids or hypotensive lipids; dopaminergic agonists and/or antagonists such as quinpirole, fenoldopam, and ibopamine; vasospasm inhibitors; vasodilators; antihypertensive agents; angiotensin converting enzyme (ACE) inhibitors; angiotensin-1 receptor antagonists such as olmesartan; microtubule inhibitors; molecular motor (dynein and/or kinesin) inhibitors; actin cytoskeleton regulatory agents such as cyctchalasin, latrunculin, swinholide A, ethacrynic acid, H-7, and Rho-kinase (ROCK) inhibitors; remodeling inhibitors; modulators of the extracellular matrix such as tert-butylhydro-quinolone and AL-3037A; adenosine receptor agonists and/or antagonists such as dicyanopyridines, N-6-cylclophexyladenosine and (R)-phenylisopropyladenosine; serotonin agonists; hormonal agents such as estrogens, estradiol, progestational hormones, progesterone, insulin, calcitonin, parathyroid hormone, peptide and vasopressin hypothalamus releasing factor; growth factor antagonists or growth factors, including, for example, epidermal growth factor, fibroblast growth factor, platelet derived growth factor, transforming growth factor beta, somatotrapin, fibronectin, connective tissue growth factor, bone morphogenic proteins (BMPs); cytokines such as interleukins, CD44, cochlin, and serum amyloids, such as serum amyloid A.

Other therapeutic agents can include neuroprotective agents such as lubezole, nimodipine and related compounds, and including blood flow enhancers, sodium channels blockers, glutamate inhibitors such as memantine, neurotrophic factors, nitric oxide synthase inhibitors; free radical scavengers or anti-oxidants; chelating compounds; apoptosis-related protease inhibitors; compounds that reduce new protein synthesis; radiotherapeutic agents; photodynamic therapy agents; gene therapy agents; genetic modulators; and dry eye medications such as cyclosporine A, delmulcents, and sodium hyaluronate.

Other therapeutic agents that can be used include: other beta-blocker agents such as acebutolol, atenolol, bisoprolol, carvedilol, asmolol, labetalol, nadolol, penbutolol, and pindolol; other corticosteroidal and non-steroidal anti-inflammatory agents such aspirin, betamethasone, cortisone, diflunisal, etodolac, fenoprofen, fludrocortisone, flurbiprofen, hydrocortisone, ibuprofen, indomethacine, ketoprofen, meclofenamate, mefenamic acid, meloxicam, methylprednisolone, nabumetone, naproxen, oxaprozin, prednisolone, prioxicam, salsalate, sulindac and tolmetin; COX-2 inhibitors like celecoxib, rofecoxib and Valdecoxib; other immune-modulating agents such as aldesleukin, adalimumab (HUMIRA®), azathioprine, basiliximab, daclizumab, etanercept (ENBREL®), hydroxychloroquine, infliximab (REMICADE®), leflunomide, methotrexate, mycophenolate mofetil, and sulfasalazine; other anti-histamine agents such as loratadine, desloratadine, cetirizine, diphenhydramine, chlorpheniramine, dexchlorpheniramine, clemastine, cyproheptadine, fexofenadine, hydroxyzine and promethazine; other anti-infective agents such as aminoglycosides such as amikacin and streptomycin; anti-fungal agents such as amphotericin B, caspofungin, clotrimazole, fluconazole, itraconazole, ketoconazole, voriconazole, terbinafine and nystatin; anti-malarial agents such as chloroquine, atovaquone, mefloquine, primaquine, quinidine and quinine; anti-mycobacterium agents such as ethambutol, isoniazid, pyrazinamide, rifampin and rifabutin; anti-parasitic agents such as albendazole, mebendazole, thiobendazole, metronidazole, pyrantel, atovaquone, iodoquinaol, ivermectin, paromycin, praziquantel, and trimatrexate; other anti-viral agents, including anti-CMV or anti-herpetic agents such as acyclovir, cidofovir, famciclovir, gangciclovir, valacyclovir, valganciclovir, vidarabine, trifluridine and foscarnet; protease inhibitors such as ritonavir, saquinavir, lopinavir, indinavir, atazanavir, amprenavir and nelfinavir; nucleotide/nucleoside/non-nucleoside reverse transcriptase inhibitors such as abacavir, ddl, 3TC, d4T, ddC, tenofovir and emtricitabine, delavirdine, efavirenz and nevirapine; other anti-viral agents such as interferons, ribavirin and trifluridiene; other anti-bacterial agents, including cabapenems like ertapenem, imipenem and meropenem; cephalosporins such as cefadroxil, cefazolin, cefdinir, cefditoren, cephalexin, cefaclor, cefepime, cefoperazone, cefotaxime, cefotetan, cefoxitin, cefpodoxime, cefprozil, ceftaxidime, ceftibuten, ceftizoxime, ceftriaxone, cefuroxime and loracarbef; other macrolides and ketolides such as azithromycin, clarithromycin, dirithromycin and telithromycin; penicillins (with and without clavulanate) including amoxicillin, ampicillin, pivampicillin, dicloxacillin, nafcillin, oxacillin, piperacillin, and ticarcillin; tetracyclines such as doxycycline, minocycline and tetracycline; other anti-bacterials such as aztreonam, chloramphenicol, clindamycin, linezolid, nitrofurantoin and vancomycin; alpha blocker agents such as doxazosin, prazosin and terazosin; calcium-channel blockers such as amlodipine, bepridil, diltiazem, felodipine, isradipine, nicardipine, nifedipine, nisoldipine and verapamil; other anti-hypertensive agents such as clonidine, diazoxide, fenoldopan, hydralazine, minoxidil, nitroprusside, phenoxybenzamine, epoprostenol, tolazoline, treprostinil and nitrate-based agents; anti-coagulant agents, including heparins and heparinoids such as heparin, dalteparin, enoxaparin, tinzaparin and fondaparinux; other anti-coagulant agents such as hirudin, aprotinin, argatroban, bivalirudin, desirudin, lepirudin, warfarin and ximelagatran; anti-platelet agents such as abciximab, clopidogrel, dipyridamole, optifibatide, ticlopidine and tirofiban; prostaglandin PDE-5 inhibitors and other prostaglandin agents such as alprostadil, carboprost, sildenafil, tadalafil and vardenafil; thrombin inhibitors; antithrombogenic agents; anti-platelet aggregating agents; thrombolytic agents and/or fibrinolytic agents such as alteplase, anistreplase, reteplase, streptokinase, tenecteplase and urokinase; anti-proliferative agents such as sirolimus, tacrolimus, everolimus, zotarolimus, paclitaxel and mycophenolic acid; hormonal-related agents including levothyroxine, fluoxymestrone, methyltestosterone, nandrolone, oxandrolone, testosterone, estradiol, estrone, estropipate, clomiphene, gonadotropins, hydroxyprogesterone, levonorgestrel, medroxyprogesterone, megestrol, mifepristone, norethindrone, oxytocin, progesterone, raloxifene and tamoxifen; anti-neoplastic agents, including alkylating agents such as carmustine lomustine, melphalan, cisplatin, fluorouracil3, and procarbazine antibiotic-like agents such as bleomycin, daunorubicin, doxorubicin, idarubicin, mitomycin and plicamycin; anti proliferative agents (such as 1,3-cis retinoic acid, 5-fluorouracil, taxol, rapamycin, mitomycin C and cisplatin); antimetabolite agents such as cytarabine, fludarabine, hydroxyurea, mercaptopurine and 5-flurouracil (5-FU); immune modulating agents such as aldesleukin, imatinib, rituximab and tositumomab; mitotic inhibitors docetaxel, etoposide, vinblastine and vincristine; radioactive agents such as strontium-89; and other anti-neoplastic agents such as irinotecan, topotecan and mitotane.

FIG. 2B is a perspective view showing a volume 191 defined by the tubular body 102 of the ocular implant 100 shown in the previous figure. The volume 191 extends along the curved longitudinal axis 116. The volume 191 can have a longitudinal radius 193, a lateral radius 195, and a generally circular lateral cross section 197.

The ocular implant 100 can be sufficiently flexible to assume a shape matching the longitudinal curvature of Schlemm's canal when the ocular implant 100 is advanced into the eye. In some embodiments, a length of the ocular implant 100 is selected so that the ocular implant 100 will extend across a pre-selected angular span when the ocular implant 100 is positioned in Schlemm's canal. Examples of pre-selected angular spans that may be suitable in some applications include 30°, 60°, 90°, 150°, and 180°.

In some embodiments, the diameter of the ocular implant 100 can range between about 0.005 inches (0.127 millimeters) and about 0.04 inches (1.016 millimeters). For example, the diameter of the ocular implant 100 can range between about 0.005 inches (0.127 millimeters) and about 0.02 inches (0.508 millimeters).

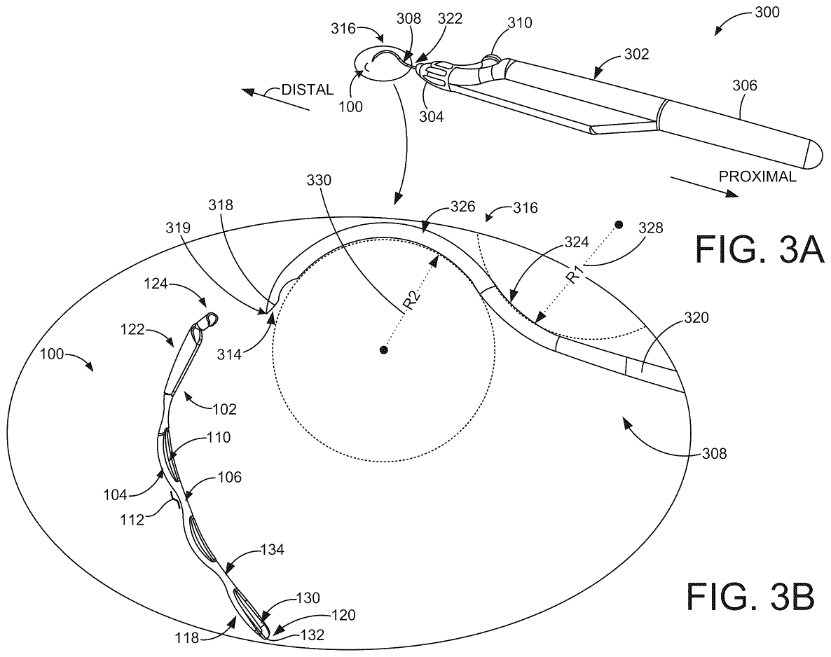

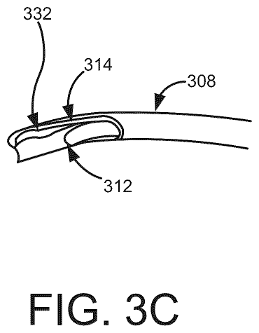

FIG. 3A is a perspective view of one embodiment of a delivery system 300 used to deliver the ocular implant 100. The delivery system 300 can be used to advance the ocular implant 100 into a target location (e.g., Schlemm's canal, the trabecular meshwork, the suprachoroidal space, the anterior chamber, etc.) in the eye of the subject.

The delivery system 300 can comprise a system housing 302, a sleeve 304, an end cap 306, and a cannula 308 extending from a distal end of the system housing 302. The delivery system 300 can also comprise a tracking wheel 310 extending through part of a wall of the system housing 302. The tracking wheel 310 can be part of a mechanism that is capable of advancing and retracting a delivery tool 312 (see FIG. 3C) of the delivery system 300. The delivery tool 312 can extend through a distal opening 314 of the cannula 308. Rotating the tracking wheel 310 can cause the delivery tool 312 to move in an axial direction within the lumen of the cannula 308. The axial direction can be in a distal direction or a proximal direction.

In the embodiment shown in FIG. 3A, the system housing 302 of the delivery system 300 can be used as a handle such that a user (e.g., a medical professional) can grip the housing 302 with one hand while the tracking wheel 310 is rotated or otherwise manipulated by one finger of the same hand to control axial advancement and retraction of the delivery tool 312. When the ocular implant 100 is grasped or clutched by, or otherwise detachably coupled to, the delivery tool 312, advancing or retracting the delivery tool 312 can also advance or retract the ocular implant 100.

FIG. 3B is an enlarged view of a distal portion 316 of the cannula 308 of the delivery system 300 and the ocular implant 100. As previously discussed, the delivery system 300 is configured to control the advancement and retraction of the ocular implant 100 through and out of the cannula 308 of the delivery system 300. For example, the ocular implant 100 can be implanted in Schlemm's canal by advancing the ocular implant 100 through a distal opening 314 of the cannula 308 while the distal opening 314 is in fluid communication with Schlemm's canal.

The cannula 308 can be sized and configured so that at least a segment of the distal portion 316 of the cannula 308 can be advanced through the trabecular meshwork of the eye and into Schlemm's canal. The cannula 308 can comprise sharp beveled edges 318 terminating at a cannula distal end 319. The beveled edges 318 and the cannula distal end 319 can be configured to cut through the trabecular meshwork and the wall of Schlemm's canal. The beveled edges 318 can be raised with respect to an inner surface of the cannula 308. In this manner, a distal end segment of the cannula 308 can be designed like a sloped trough that surrounds the ocular implant 100 as the ocular implant 100 is advanced out of the distal opening 314 of the cannula 308 by the delivery tool 312.

The cannula 308 can also comprise a generally straight tubular portion 320 at a proximal end 322 of the cannula 308 (the end that is coupled to the system housing 302) and a curved distal portion 316 in between the straight tubular portion 320 and the distal opening 314.

The curved distal portion 316 of the cannula 308 can comprise a proximal curved bend 324 and a distal curved bend 326. The proximal curved bend 324 can be disposed in between the generally straight tubular portion 320 and the distal curved bend 326. The proximal curved bend 324 can be bent in a different direction than the distal curved bend 326.

In some embodiments, the proximal curved bend 324 can be bent in a radially outward direction or where a concavity of the bend faces a dorsal side of the system housing 302 or the side with the tracking wheel 310. In these embodiments, the distal curved bend 326 can be bent in a radially inward direction or where a concavity of the bend faces a ventral side or underside of the system housing 302.

The proximal curved bend 324 can have a proximal bend radius 328 (also shown as R1 in FIG. 3B) and the distal curved bend 326 can have a distal bend radius 330 (also shown as R2 in FIG. 3B).

In some embodiments, the proximal bend radius 328 or R1 can be substantially equivalent to the distal bend radius 330 or R2. For example, the distal bend radius 330 or R2 can be between about 3.0 mm and 4.0 mm. As a more specific example, the distal bend radius 330 or R2 can be about 3.4 mm. The proximal bend radius 328 or R1 can be between about 3.0 mm and 4.0 mm. As a more specific example, the proximal bend radius 328 or R1 can be about 3.4 mm. In other embodiments, the proximal bend radius 328 or R1 can be greater than the distal bend radius 330 or R2.

In some embodiments, the curved distal portion 316 of the cannula 308 can be shaped substantially as a shepherd's hook or a top portion of a question mark.

One technical problem faced by the applicant is how to design a cannula for an ocular implant delivery system such that the approach angle of the cannula with respect to Schlemm's canal is as near tangential as possible. This is because the surgeon would not be able to release the ocular implant from the cannula if the distal tip of the cannula does not pierce the trabecular meshwork into Schlemm's canal tangentially. One technical solution discovered and developed by the applicant is the cannula 308 depicted and described herein where the distal portion 316 of the cannula 308 comprises a proximal curved bend 324 and a distal curved bend 326 and where the proximal curved bend 324 is bent in a different direction than the distal curved bend 326. Moreover, a proximal bend radius 328 of the proximal curved bend 324 is greater than a distal bend radius 330 of the distal curved bend 326. The applicant also discovered that another added benefit to the cannula 308 depicted and described herein is that the unique shepherd's hook shape of the cannula 308 allows the cannula 308 to use an intraocular incision made for cataract surgeries for entry into the eye for the procedure disclosed herein (i.e., deploying the ocular implant 100 into Schlemm's canal).

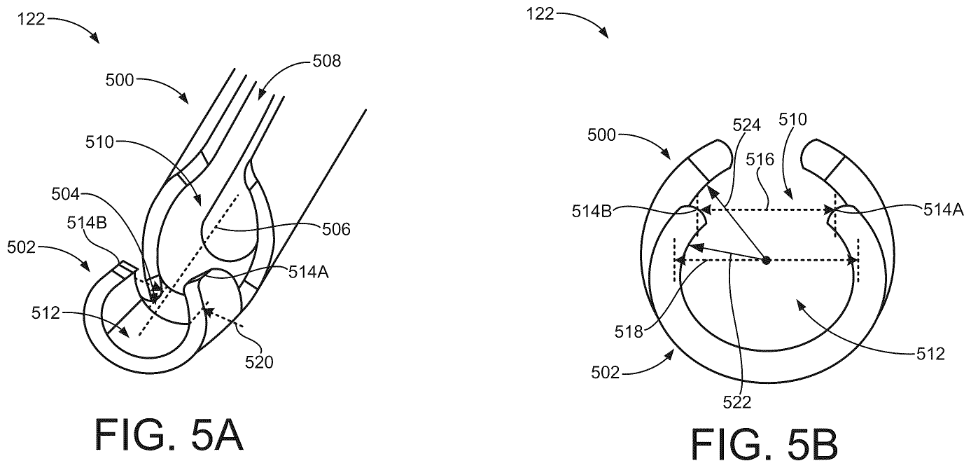

FIG. 3C illustrates that the delivery tool 312 of the delivery system 300 can extend through and partially out of the distal opening 314 of the cannula 308. As shown in FIG. 3C, the delivery tool 312 can comprise an interlocking portion 332 that is configured to engage with a complementary interlocking feature defined along the proximal portion 122 of the ocular implant 100 (see FIG. 5A). Rotating the tracking wheel 310 can cause the delivery tool 312 and the ocular implant 100 to move along a path defined by the cannula 308.

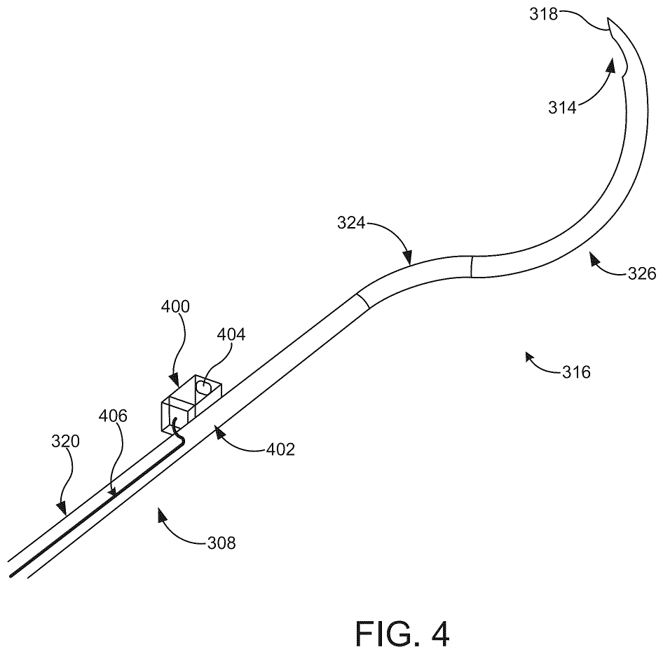

FIG. 4 is a perspective view of one embodiment of the cannula 308 of the delivery system 300 comprising a camera 400 coupled to the cannula 308. The camera 400 can allow the surgeon to directly observe the surgical field during an implantation procedure.

In some embodiments, the camera 400 can be coupled to an exterior surface 402 of the cannula 308. The camera 400 can be coupled to the exterior surface 402 of the cannula 308 via an adhesive, one or more mechanical fasteners, or a combination thereof.

In some embodiments, at least part of the camera 400 can be positioned within the cannula 308 and an aperture or opening defined along the exterior surface 402 of the cannula 308 can expose part of the camera 400 (such as a lens 404 of the camera 400). In certain embodiments, the lens 404 of the camera 400 can be a glass lens.

In certain embodiments, the camera 400 can be coupled to a substantially straight shaft of the cannula 308 or the straight tubular portion 320 of the cannula 308. For example, the camera 400 can be coupled to the exterior surface 402 of the substantially straight shaft or the straight tubular portion 320 of the cannula 308

In other embodiments, the camera 400 can be coupled to the proximal curved bend 324 of the cannula 308.

The camera 400 can be positioned or otherwise coupled to the cannula 308 in a way that allows the camera 400 to capture a deployment of the ocular implant 100 out of the distal opening 314 of the cannula 308. For example, the camera lens 404 of the camera 400 can be positioned to face the distal opening 314 of the cannula 308.

In some embodiments, the camera 400 can comprise a complementary metal-oxide-semiconductor (CMOS) image sensor. In certain embodiments, the CMOS image sensor can have a sensor width of between about 0.65 mm and 0.75 mm (or about 0.70 mm). The camera 400 can capture videos at between about 30 frames per second and 60 frames per second.

In some embodiments, the lens 404 of the camera 400 can have a field-of-view of between about 100 degrees and 130 degrees (or about 120 degrees). The camera 400 can also have a focal range of between about 3.0 mm and 30 mm.

In some embodiments, the camera 400 can be communicatively coupled to a camera control unit or a computing device via a wired cable connection 406. For example, the wired cable connection 406 can be an ultrafine cable having a cable length of at least 3.0 meters.

Although FIG. 4 illustrates the camera 400 coupled to a cannula 308 comprising a proximal curved bend 324 and a distal curved bend 326, it is contemplated by this disclosure that the camera 400 disclosed herein can be coupled to a cannula comprising only one curved bend. For example, it is contemplated by this disclosure that the camera 400 disclosed herein can be coupled to any of the cannulas disclosed in U.S. Pat. No. 11,197,779, the content of which is incorporated herein by reference in its entirety.

FIGS. 5A and 5B are close-up perspective and front views, respectively, of a proximal portion 122 of one embodiment of the ocular implant 100. The proximal portion 122 can comprise a substantially-tubular portion 500 and a pair of tool engagement ears 502 coupled to the substantially-tubular portion 500 by a narrow connecting portion 504. The proximal portion 122 of the ocular implant 100 can be configured to interlock, mate, or detachably engage with a complementary portion of a delivery tool 312 translatable within a cannula 308 of a delivery system 300 (see, e.g., FIGS. 3A and 3B). For example, a distal interlocking portion 332 of the delivery tool 312 can be designed to interlock or hook onto at least part of the proximal portion 122 of the ocular implant 100.

The pair of tool engagement ears 502 can be curled laterally inward toward one another. For example, the pair of tool engagement ears 502 can be curled laterally inward toward a medial line 506 bisecting the proximal portion 122.

The substantially-tubular portion 500 can have an elongated gap 508 extending along a length of the substantially-tubular portion 500. The substantially-tubular portion 500 can also define a tubular-shaped lumen 510 extending longitudinally through the substantially-tubular portion 500. The tubular-shaped lumen 510 can be in fluid communication with a trough-shaped cavity 512 defined by the pair of tool engagement ears 502.

As shown in FIGS. 5A and 5B, the pair of tool engagement ears 502 can comprise a first ear terminal end 514A and a second ear terminal end 514B. The first ear terminal end 514A can be separated from the second ear terminal end 514B by a minimum separation distance 516. The tubular-shaped lumen 510 can have a lumen diameter 518. The minimum separation distance 516 can be less than the lumen diameter 518.

In some embodiments, the minimum separation distance 516 can be between about 0.15 mm and about 0.25 mm. In these and other embodiments, the lumen diameter 518 can be between about 0.28 mm and 0.32 mm (or about 0.30 mm).

As shown in FIG. 5A, the narrow connecting portion 504 can have a minimum connecting portion width 520. The minimum connecting portion width 520 can be smaller than the minimum separation distance 516 and the lumen diameter 518.

Each of the tool engagement ears 502 can comprise an ear radius of curvature 522 along part of the tool engagement ear 502. The substantially-tubular portion 500 can comprise a tube radius of curvature 524. The ear radius of curvature 522 can be different from the tube radius of curvature 524. For example, the tube radius of curvature 524 can be greater than the ear radius of curvature 522.

In some embodiments, the tube radius of curvature 524 can be between about 0.14 mm and about 0.26 mm (or about 0.15 mm). In these and other embodiments, the ear radius of curvature 522 can be between about 0.08 mm and 0.12 mm.

One technical problem faced by the applicant is how to prevent the ocular implant 100 from being seized or locked up in between the inner wall of the cannula 308 and the outer wall of the delivery tool 312 when the thin-wall of the proximal portion 122 of the ocular implant 100 inadvertently slides in between the inner wall of the cannula 308 and the outer wall of the delivery tool 312. One technical solution discovered and developed by the applicant is to design the proximal portion 122 of the ocular implant 100 to include a pair of tool engagement ears 502 curled laterally inward toward a medial line 506 bisecting the proximal portion 122. When the proximal portion 122 of the ocular implant 100 is implemented in this manner, the proximal portion 122 of the ocular implant 100 is less likely to slide in between the inner wall of the cannula 308 and the outer wall of the delivery tool 312 to cause the ocular implant 100 to be seized by or locked up with the cannula 308 and the delivery tool 312.

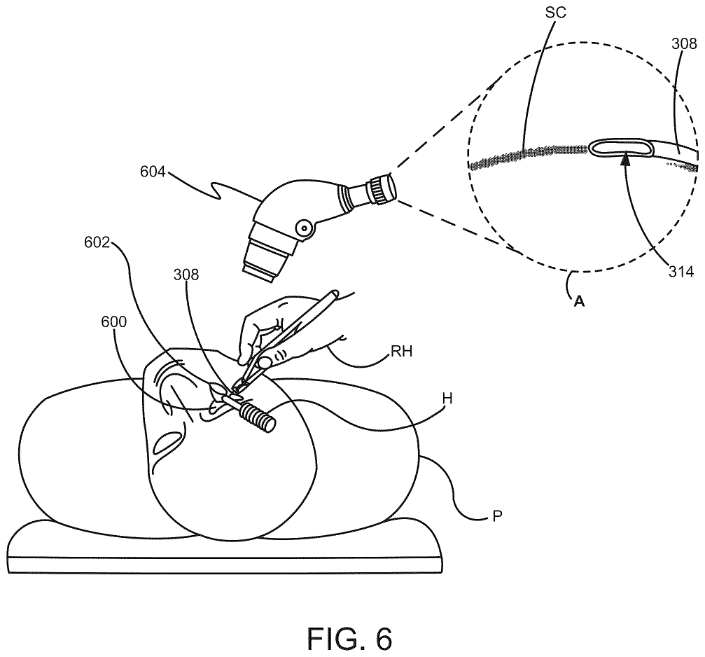

FIG. 6 is a stylized representation of part of a procedure for deploying the ocular implant 100. In the procedure depicted in FIG. 6, a physician is treating an eye 600 of a patient P. The physician is holding the system housing 302 or handle portion of the delivery system 300 in their right hand RH. The physician's left hand (not shown) can be used to hold the handle H of a gonio lens 602. Alternatively, some physicians may prefer holding the handle portion of the delivery system 300 in their left hand and the gonio lens handle H in their right hand RH.

The physician can view the interior of the anterior chamber using gonio lens 602 and a microscope 604. Close-up view A of FIG. 6 is a stylized illustration of what can be seen by the physician during the procedure. A distal portion of cannula 308 is visible in close-up view A. A shadow-like line indicates the location of Schlemm's canal (SC) which is lying under various tissue (e.g., the trabecular meshwork) that surround the anterior chamber of the eye 600. A distal opening 314 of the cannula 308 is positioned near Schlemm's canal.

In some embodiments, a method of deploying the ocular implant 100 (or delivering the ocular implant 100 as part of an implantation procedure) can comprise inserting a part of the cannula 308 through an incision in the eye of the subject. The method can also comprise placing the distal opening 314 of the cannula 308 into fluid communication with Schlemm's canal of the eye and advancing an implant body 102 of the ocular implant 100 out of the distal opening 314 of the cannula 308 into Schlemm's canal.

In certain embodiments, the ocular implant 100 can be advanced through a cannula distal portion 316 using a delivery tool 312 detachably coupled to the ocular implant 100. The cannula distal portion 316 can comprise a proximal curved bend 324 and a distal curved bend 326. The proximal curved bend 324 can be bent in a different direction than the distal curved bend 326.

In some embodiments, the method can also comprise capturing a video of the ocular implant 100 advancing out of the distal opening 314 of the cannula 308 using a camera 400 coupled to the cannula 308.

As previously discussed, insertion of the ocular implant 100 into Schlemm's canal can facilitate the flow of aqueous humor out of the anterior chamber of the eye.

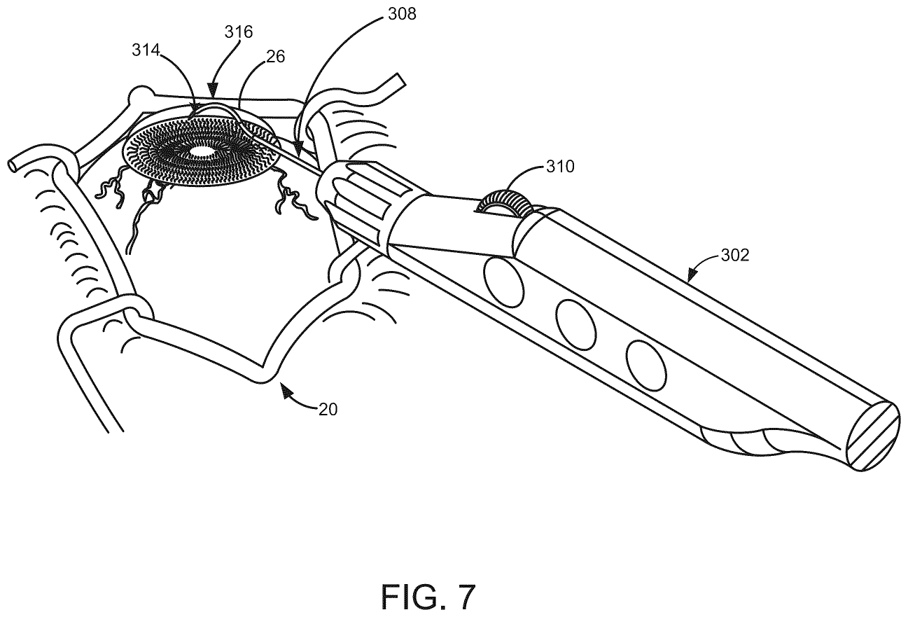

FIG. 7 illustrates another stylized representation of the procedure for deploying the ocular implant 100. As shown in FIG. 7, the cannula 308 of the delivery system 300 is shown extending through the cornea 26 of the eye 20. A distal portion 316 of the cannula 308 is disposed inside the anterior chamber defined by the cornea 26 of the eye 20. The cannula 308 is configured so that the distal opening 314 of the cannula 308 is placed in fluid communication with Schlemm's canal.

As previously discussed, the delivery system 300 can comprise a mechanism (e.g., the tracking wheel 310 and the delivery tool 312) capable of advancing and retracting the ocular implant 100 within the cannula 308. The ocular implant 100 can be placed in Schlemm's canal of the eye 20 by advancing the ocular implant 100 through the distal opening 314 of the cannula 308 while the distal opening 314 is in fluid communication with Schlemm's canal.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An ocular implant, comprising:
an implant body extending in a curved configuration and configured to lower an intraocular pressure of an eye when implanted within Schlemm's canal of the eye, wherein the implant body forms an arc, wherein the implant body comprises:
a distal portion having a distal end and a proximal portion having a proximal end,
a plurality of openings disposed in between the distal end and the proximal end, and
a plurality of supporting frames and spines connecting the supporting frames, wherein each of the openings is surrounded by at least one of the supporting frames,
wherein the distal portion comprises a distal supporting frame configured as an open trough having a trough nadir at the distal end, and
wherein the trough nadir is positioned radially outward of the spines.

2. The ocular implant of claim 1, wherein the distal supporting frame comprises a first distal frame strut and a second distal frame strut, wherein the first distal frame strut and the second distal frame strut form sides of the open trough.

3. The ocular implant of claim 1, wherein the open trough has a trough concavity, wherein the trough concavity faces a trabecular meshwork of the eye when the ocular implant is implanted within Schlemm's canal.

4. The ocular implant of claim 1, wherein the distal portion comprises a distal opening surrounded by the distal supporting frame, wherein the distal opening comprises an opening distal end and an opening proximal end, wherein the opening distal end of the distal opening is radially outward of the opening proximal end of the distal opening.

5. The ocular implant of claim 1, wherein the openings comprise a distal opening surrounded by the distal supporting frame and an intermediate opening positioned proximal to the distal opening, wherein the distal opening is defined by a distal opening contour, wherein the intermediate opening is defined by an intermediate opening contour, wherein the distal opening contour is shaped differently from the intermediate opening contour.

6. The ocular implant of claim 1, wherein the supporting frames further comprise an intermediate supporting frame positioned proximally of the distal supporting frame, wherein a shape of the intermediate supporting frame is different from the shape of the distal supporting frame.

7. The ocular implant of claim 1, wherein the proximal portion comprises a substantially-tubular portion and a pair of tool engagement ears coupled to the substantially-tubular portion by a narrow connecting portion, wherein the pair of tool engagement ears are curled laterally inward toward one another, wherein the proximal portion is configured to interlock with a complementary portion of a delivery tool.

8. The ocular implant of claim 7, wherein the substantially-tubular portion has an elongated gap extending along a length of the substantially-tubular portion, and wherein the substantially-tubular portion defines a tubular-shaped lumen extending therethrough.

9. The ocular implant of claim 8, wherein the pair of tool engagement ears comprise a first ear terminal end and second ear terminal end, wherein the first ear terminal end is separated from the second ear terminal end by a minimum separation distance, wherein the tubular-shaped lumen has a lumen diameter, wherein the minimum separation distance is less than the lumen diameter.

10. The ocular implant of claim 9, wherein the narrow connecting portion has a minimum connecting portion width, wherein the minimum connecting portion width is smaller than the minimum separation distance and the lumen diameter.

11. The ocular implant of claim 7, wherein each of the tool engagement ears comprises an ear radius of curvature, wherein the substantially-tubular portion comprises a tube radius of curvature, wherein the ear radius of curvature is different from the tube radius of curvature.

12. The ocular implant of claim 7, wherein the pair of tool engagement ears are curled laterally inward toward a medial line bisecting the proximal portion.

13. The ocular implant of claim 1, wherein the ocular implant is configured to be deployed out of a cannula, wherein the cannula comprises a proximal curved bend and a distal curved bend, wherein the proximal curved bend is bent in a different direction than the distal curved bend.

14. The ocular implant of claim 13, wherein the distal curved bend has a bend radius of curvature of between 3.0 mm and 4.0 mm.

15. The ocular implant of claim 1, wherein the ocular implant is configured to be deployed out of a cannula, wherein the cannula comprises a camera coupled to at least part of the cannula.

16. An ocular implant, comprising:
an implant body extending in a curved configuration and configured to lower an intraocular pressure of an eye when implanted within Schlemm's canal of the eye, wherein the implant body forms an arc, wherein the implant body comprises:
a proximal portion having a proximal end and a distal portion having a distal end,
a plurality of openings disposed in between the distal end and the proximal end, and
a plurality of supporting frames and spines connecting the supporting frames, wherein each of the openings is surrounded by at least one of the supporting frames,
wherein the proximal portion comprises a substantially-tubular portion and a pair of tool engagement ears coupled to the substantially-tubular portion by a narrow connecting portion, wherein the pair of tool engagement ears are curled laterally inward toward one another, and
wherein the proximal portion is configured to interlock with a complementary portion of a delivery tool.

17.-28. (canceled)

29. A delivery system, comprising:
a system housing;
a cannula extending from the system housing, wherein the cannula comprises a cannula distal portion comprising a proximal curved bend and a distal curved bend, wherein the proximal curved bend is bent in a different direction than the distal curved bend; and
a delivery tool housed at least partially within the cannula and translatable therethrough, wherein the delivery tool is configured to detachably engage with an ocular implant, wherein the ocular implant comprises:
an implant body extending in a curved configuration and configured to lower an intraocular pressure of an eye when implanted within Schlemm's canal of the eye, wherein the implant body comprises:
a distal portion having a distal end and a proximal portion having a proximal end,
a plurality of openings disposed in between the distal end and the proximal end, and
a plurality of supporting frames and spines connecting the supporting frames, wherein each of the openings is surrounded by at least one of the supporting frames.

30.-35. (canceled)

36. A delivery system, comprising:
a system housing;
a cannula extending from the system housing;
a camera coupled to the cannula; and
a delivery tool housed at least partially within the cannula and translatable therethrough, wherein the delivery tool is configured to detachably engage with an ocular implant, wherein the ocular implant comprises:
an implant body extending in a curved configuration and configured to lower an intraocular pressure of an eye when implanted within Schlemm's canal of the eye, wherein the implant body comprises:
a distal portion having a distal end and a proximal portion having a proximal end,
a plurality of openings disposed in between the distal end and the proximal end, and
a plurality of supporting frames and spines connecting the supporting frames, wherein each of the openings is surrounded by at least one of the supporting frames.

37.-48. (canceled)

49. A method of deploying an ocular implant, comprising:
inserting a part of a cannula through an incision in an eye;
placing a distal opening of the cannula into fluid communication with Schlemm's canal of the eye;
advancing an implant body of the ocular implant through a cannula distal portion using a delivery tool detachably coupled to the ocular implant, wherein the cannula distal portion comprises a proximal curved bend and a distal curved bend, wherein the proximal curved bend is bent in a different direction than the distal curved bend; and
advancing the ocular implant out of the distal opening of the cannula and into Schlemm's canal.

50. A method of deploying an ocular implant, comprising:
inserting a part of a cannula through an incision in an eye;
placing a distal opening of the cannula into fluid communication with Schlemm's canal of the eye;
advancing an implant body of the ocular implant out of a distal opening of the cannula and into Schlemm's canal; and
capturing a video of the ocular implant advancing out of the distal opening of the cannula using a camera coupled to the cannula.
